(19) United States
(12) Patent Application Publication
(10) Pub. No.: US 2012/0213867 A1
(43) Pub. Date: Aug. 23, 2012

(54) RAF INHIBITORS FOR THE TREATMENT OF THYROID CANCER

(75) Inventor: Darrin Douglas Stuart, Pleasant Hill, CA (US)

(73) Assignee: Novartis AG

(21) Appl. No.: 13/457,273

(22) Filed: Apr. 26, 2012

Related U.S. Application Data
(63) Continuation of application No. 12/600,720, filed on Nov. 18, 2009.
(60) Provisional application No. 60/939,625, filed on May 23, 2007.

(51) Int. Cl. A61K 31/4439; A61K 33/24; A61P 35/00

(52) U.S. Cl. 424/649; 514/338

(57) ABSTRACT

The invention relates to the use of an Raf inhibitor for the manufacture of pharmaceutical compositions for the treatment of thyroid cancer, more specifically papillary thyroid cancer (PTC); the use of a Raf inhibitor in the treatment of thyroid cancer, more specifically PTC; a method of treating warm-blooded animals including mammals, especially humans, suffering from thyroid cancer, more specifically PTC, by administering to a said animal in need of such treatment a dose effective against said disease of an Raf inhibitor. The invention also relates to the use of a Raf inhibitor in combination with a platin compound for the treatment of thyroid cancer, more specifically papillary thyroid cancer.

### FIELD OF THE INVENTION

The invention relates to the use of a Rat inhibitor for the manufacture of pharmaceutical compositions for the treatment of thyroid cancer, more specifically papillary thyroid cancer (PTC); the use of a Raf inhibitor in the treatment of thyroid cancer, more specifically PIC; a method of treating warm-blooded animals including mammals, especially humans, suffering from thyroid cancer, more specifically PTC, by administering to a said animal in need of such treatment a dose effective against said disease of a Raf inhibitor. The invention also relates to the use of a Raf inhibitor in combination with a platin compound for the treatment of thyroid cancer, more specifically papillary thyroid cancer.

### BACKGROUND OF THE INVENTION

Thyroid cancer is a relatively rare disease comprising approximately 1% of all new cancer diagnoses each year (26.000 cases per year in the US). The most prevalent sub-type is PTC which makes up approximately 80% of all cases. While the majority of these patients are cured by surgery followed by adjuvant ¹³¹I-radioiodine therapy, some do not respond and for these patients there are few treatment options.

Genetic characterization of PTC suggests that there are opportunities for targeted therapies to impact this disease and of particular interest are targets within the Ras/Raf/MAPK pathway, Up to 70% of PIG express a mutant form of B-Raf (B-Raf^{V600E}){Chiloeches, 2006 #270; Cohen, 2003 #287}. B-Raf is normally activated by Ras and functions in this pathway to transmit proliferation and survival signals from cell surface receptors, through phosphorylation and activation of MEK. However. B-Raf^{V600E} does not require activation by Ras and constitutively activates the pathway, promoting dysregulated proliferation and suppressing apoptosis.

The importance of this pathway in PTC is emphasized by the observation that up to 30% of these tumors express a mutant form of the receptor which is caused by genomic rearrangement leading to constitutive activity the receptor tyrosine kinase activity and activation of the MAPK pathway {Viglletto, 1995 #288}. Thus, the vast majority of PTC tumors appear to activate the MAPK pathway through B-Raf or RET mutations and there is a potential therapeutic benefit from agents which target RET or B-Raf. Therefore, there is a need to develop novel treatment methods.

### SUMMARY OF THE INVENTION

Surprisingly, it was now found that Raf inhibitors treat PTC. Hence, the invention relates to the use of a Raf inhibitor for the preparation of a medicament for the treatment of PTC. The invention also relates to the use of a Raf inhibitor in the treatment of PTC. The invention relates to a method of treating warm-blooded animals including mammals, especially humans, suffering from PTC by administering to a said animal in need of such treatment a dose effective against said disease of a Raf inhibitor or a pharmaceutically acceptable salt thereof.

### DETAILED DESCRIPTION OF THE INVENTION

The Raf inhibitors are substituted benzimidazole compounds having the following formula (i):

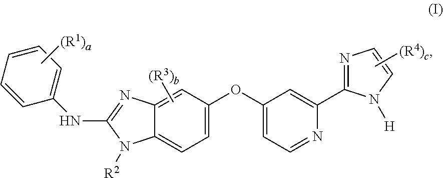

wherein
each Fe is independently selected from hydroxy, halo, C₁₋₆alkyl, C₁₋₆alkoxy, (C₁₋₆alkyl)sulfanyl, (C₁₋₆alkyl)sulfonyl, cycloalkyl, heterocycloalkyl, phenyl and heteroaryl;
R² is C₁₋₆alkyl or halo(C₁₋₆alkyl);
each R³ is independently selected from halo, C₁₋₆alkyl and C₁₋₆alkoxy;
each R⁴ is independently selected from hydroxy, C₁₋₆alkoxy, halo, heterocycloalkylcarbonyl, carboxyl, (C₁₋₆,alkoxy)carbonyl, aminocarbonyl, C₁₋₆alkylaminocarbonyl, carbonitrile, cycloalkyl, heterocycloalkyl, phenyl and heteroaryl;
wherein R¹, R², R³ and R⁴ may be optionally substituted with one or more substituents independently selected from hydroxy, halo, C₁₋₆alkyl, halo(C₁₋₆alkyl), C₁₋₆alkoxy and halo(C₁₋₆alkoxy):
a is 1, 2, 3, 4 or 5;
b is 0, 1, 2 or 3; and
c is 1 or 2;
   or a tautomer, stereoisomer, polymorph, ester, metabolite, or prodrug thereof or a pharmaceutically acceptable salt of the compound, tautomer, stereoisomer, polymorph, ester, metabolite or prodrug.

In other embodiments, new substituted benzimidazole compounds are provided of the formula (II);

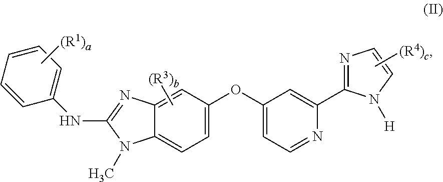

wherein
each R¹ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl and heteroaryl;
each R³ is independently selected from halo, C₁₋₆alkyl and C₁₋₆alkoxy;
each R⁴ is independently selected from hydroxy, C₁₋₆aikyl, C₁₋₆alkoxy, halo, carboxyl, (C₁₋₆alkoxy)carbonyl, aminocarbonyl, carbonitrile, cycloalkyl, heterocycioalkyl, heterocycloalkylcarbonyl, phenyl and heteroaryl;
wherein R¹, R², R³ and R⁴ may be optionally substituted with one or more substituents independently selected from hydroxy, halo, C₁₋₆alkyl and C₁₋₆alkoxy;
a is 1, 2, 3, 4 or 5;
b is 0, 1, 2 or 3; and
c is 1 or 2;
   or a tautomer, stereoisomer, polymorph, ester, metabolite, or prodrug thereof or a pharmaceutically acceptable salt of the compound, tautomer, stereoisomer, polymorph, ester, metabolite or prodrug.

In other embodiments. new substituted benzimidazole compounds are provided of the formula (III).

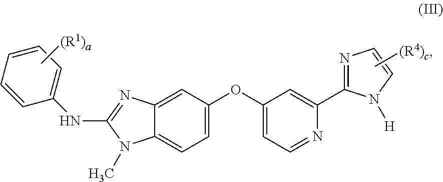

wherein
each R¹ is independently selected from C₁₋₆alkyl, C₁₋₆alkoxy, hydroxy, halo, (C₁₋₆alkyl)sulfanyl, (C₁₋₆ alkyl)sulfonyl, cycloalkyl, heterocycloalkyl, phenyl and heteroaryl;
each R⁴ is independently selected from hydroxy, C₁₋₆alkoxy, halo, carboxyl, (C₁₋₆alkoxy)carbonyl, aminocarbonyl, carbonitrile, cycloalkyl, heterocycloalkyl, heterocycloalkylcarbonyl, phenyl and heteroaryl; wherein R¹ and R⁴ may be optionally substituted with one or more substituents independently selected from hydroxy, halo, Cᵢ,ₑalkyl and C₁.,₆alkoxy;
a is 1, 2, 3, 4 or 5; and
c is 1 or 2;
   or a tautomer, stereoisomer, polymorph, ester, metabolite, or prodrug thereof or a pharmaceutically acceptable salt of the compound, tautomer, stereoisomer, polymorph, ester, metabolite or prodrug.

Also disclosed are compounds of the following formula (IV):

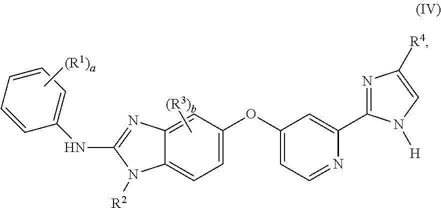

wherein
each R¹ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl and heteroaryl;
R² is C₁₋₆alkyl or halo(C₁₋₆alkyl);
each R³ is independently selected from halo, C₁₋₆alkyl and C₁₋₆alkoxy;
each R⁴ is independently selected from hydroxy, C₁₋₆alkyl, C₁₋₆alkoxy, halo, carboxyl, (C₁₋₆alkoxy)carbanyl, aminocarbonyl, C₁₋₆alkylaminocarbonyl, carbonitrile, carbonitrile(C₁₋₆alkyl), cycloalkyl, heterocycloalkyl, heterocycloalkyl(C₁₋₆alkyl), heterocycloalkylcarbonyl, phenyl and heteroaryl;
wherein R¹, R², R³ and R⁴ may be optionally substituted with one or more substituents independently selected from hydroxy, halo, C₁₋₆alkyl and C₁₋₆alkoxy;
a is 1, 2, 3, 4 or 5: and
b is 0, 1, 2 or 3;
   or a tautomer, stereoisomer, polymorph, ester, metabolite, or prodrug thereof or a pharmaceutically acceptable salt of the compound, tautomer, stereoisomer, polymorph, ester, metabolite or prodrug.

In other embodiments, new substituted benzimidazole compounds are provided of formulae (I)-(IV), wherein each R¹ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, trifluoromethylsulfanyl, piperidinyl, C₁₋₆alkylpiperidinyl. piperazinyl, C₁₋₆alkyipiperazinyl, tetrahydrofuranyl, pyridinyl and pyrimidinyl. In other embodiments, new substituted benzimidazole compounds are provided of formulae (1)-(IV), wherein a is 1 or 2, and at least one R¹ is halo(C₁₋₆alkyl), such as trifluoromethyl. In other embodiments, new substituted benzimidazole compounds are provided of formulae (I) and (IV), wherein R² is C₁₋₆alkyl, such as, e.g., methyl or ethyl. In further embodiments, new substituted benzimidazole compounds are provided of formulae (I), (II) and (IV), wherein b is 0, and thus R³ is not present, In alternate embodiments, new substituted benzimidazole compounds are provided of formulae (1)-(IV), wherein b is 1, and R³ is C₁₋₆alkoxy, such as, e.g., methoxy. In yet further embodiments, new substituted benzimidazole compounds are provided of formulae (I)-(III), wherein c is 1 or 2, and at least one R⁴ is halo(C₁₋₆alkyl), such as, e.g., trifluoromethyl.

“Alkyl” refers to saturated hydrocarbyl groups that do not contain heteroatoms and includes straight chain alkyl groups, such as methyl, ethyl, propyl, butyl, pentyl, hexyl, heptyl, octyl, nonyl, decyl, undecyl, dodecyl and the like. Alkyl also includes branched chain isomers of straight chain alkyl groups, including but not limited to, the following which are provided by way of example: —CH(CM₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)—CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃) ₃, —CH(CH₃)CH₂CH(CH₃)₂, —CH(CH₃)CH(CH₃)CH(CH₃)₂, —CH(CH₂CH₃)CH(CH₃)(CH₃)(CH₂CH₃) and others. Thus, alkyl groups include primary alkyl groups, secondary alkyl groups and tertiary alkyl groups. The phrase “C₁₋₁₂alkyl” refers to alkyl groups having from one to twelve carbon atoms. The phrase “C₁₋₆alkyl” refers to alkyl groups having from one to six carbon atoms.

“Alkenyl” refers to straight or branched hydrocarbyl groups having from 2-6 carbon atoms and preferably 2-4 carbon atoms and having at least 1 and preferably from 1-2 sites of vinyl (>C=C<) unsaturation. Such groups are exemplified, e.g., by vinyl, allyl and but-3-en-1-yl. Included within this term are the cis and trans isomers or mixtures of these isomers.

“Alkoxy” refers to RO—, wherein R is an alkyl group. The phrase “C₁₋₆alkoxy”, as used herein, refers to RO—, wherein R is a C₁₋₆alkyl group. Representative examples of C₁₋₆alkoxy groups include methoxy, ethoxy, t-butoxy and the like.

“(C₁₋₆alkoxy)carbonyl” refers to ester —C(═O)—OR, wherein R is C₁₋₆alkyl.

“Amidino” refers to the group —C(═NH)NH₂. “Amidine” refers to a compound containing such a group.

“Aminocarbonyl” refers herein to the group —C(O)—NH₂.

“C₁₋₆alkylaminocarbonyl” refers to the group —C(O)—NRR′, where R is C₁₋₆alkyl and R′ is selected from hydrogen and C₁₋₆alkyl.

“Carbonyl” refers to the divalent group —C(O)—.

“Carboxyl” refers to —C(═O)—OH,

“Cyano” “carbonitrile” or “nitrile” refers to —CN. “Carbonitrile(C₁₋₆alkyl)” refers to C₁₋₆alkyl substituted with —CN,

“Cycloalkyl” refers to a mono- or polycyclic alkyl substituent. Typical cycloalkyl groups have from 3-8 carbon ring atoms. Representative cycloalkyl groups include cyclopropyl, cyclobutyl, cyclopentyl, cyciohexyl, cycioheptyl and cyclooctyl.

“Halogen” or “halo” refers to chloro, bromo, fluoro and iodo groups.

“Halo(C₁₋₆alkyl)” refers to a C₁₋₆alkyl radical substituted with one or more halogen atoms, preferably one to five halogen atoms. A more preferred halo(C₁₋₆alkyl) group is trifluoromethyl.

“Halo(C₁₋₆alkyl)phenyl” refers to a phenyl group substituted with a halo(C₁₋₆alkyl) group.

“Halo(C₁₋₆alkoxy)” refers to an alkoxy radical substituted with one or more halogen atoms, preferably one to five halogen atoms. A more preferred halo(C₁₋₆alkoxy) group is trifluoromethoxy.

“Halo(C₁₋₆alkyl)sulfonyl” and “halo(C₁₋₆alkyl)sulfanyl” refer to substitution of sulfonyl and sulfonyl groups with halo(C₁₋₆alkyl) groups, wherein sulfonyl and sulfonyl are as defined herein.

“Heteroaryl” refers to an aromatic group having from 1-4 heteroatoms as ring atoms in an aromatic ring with the remainder of the ring atoms being carbon atoms, Suitable heteroatoms employed in compounds of the present invention are nitrogen, oxygen and sulfur, wherein the nitrogen and sulfur atoms may be optionally oxidized. Exemplary heteroaryl groups have 5-14 ring atoms and include, e.g., benzimidazolyl, benzothiazolyl, benzoxazolyl, diazapinyl, (uranyl_{:} pyrazinyl, pyrazolyl, pyridyl, pyridazinyl, pyrimidinyl, pyrroyl, oxazolyl, isoxazolyl, imidazolyl, indolyl, indazolyl, quinolinyl, isoquinolinyl, quinazolinyl, quinoxalinyl, thiazolyl, thienyl and triazolyl.

“Heterocycloalkyl” refers herein to cycloalkyl substituents that have from 1-5, and more typically from 1-2 heteroatoms in the ring structure. Suitable heteroatoms employed in compounds of the present invention are nitrogen, oxygen and sulfur, wherein the nitrogen and sulfur atoms may be optionally oxidized. Representative heterocycloalkyl moieties include, e.g., morpholino, piperazinyl, piperidinyl and the like.

“(C₁₋₆alkyl)heterocycloalkyl” refers to a heterocycloalkyl group substituted with a C₁₋₆alkyl group.

“Heterocycloalkyl(C₁₋₆alkyl)” refers to C₁₋₆alkyl substituted with heterocycloalkyl.

“Heterocycloalkyloarbonyl” refers herein to the group —C(O)—R¹⁰, where R¹⁰ is heterocycloalkyl.

“(C₁₋₆alkyl)heterocycloalkylcarbonyl” refers to the group —C(O)—R¹¹, where R¹¹ is (C₁₋₆alkyl)heterocycloalkyl.

“Hydroxy” refers to —OH.

“Hydroxy(C₁₋₆alkyl)” refers to a C₁₋₆alkyl group substituted with hydroxy.

“Hydroxy(C₁₋₈alkylaminocarbonyl)” refers to a C₁₋₆alkylaminocarbonyl group substituted with hydroxy.

“imidate” or “imidate ester” refers to the group —C(═NH)O— or to a compound containing such a group. Imidate esters include, e.g., the methyl ester imidate —C(═NH)OCH₃.

“Nitro” refers to —NO₂.

“Sulfonyl” refers herein to the group —SO₂—.

“Sulfanyl” refers herein to the group —S—. “Alkylsulfonyl” refers to a substituted sulfonyl of the structure —SO₂R¹² in which R¹² is alkyl. “Alkylsulfanyl” refers to a substituted sulfanyl of the structure —SR¹² in which R¹² is alkyl. Alkylsulfonyl and alkylsulfanyl groups employed in compounds of the present invention include (C₁₋₆alkyl)sulfonyl and (C₁₋₆alkyl)sulfanyl. Thus, typical groups include, e.g., methylsulfonyl and methylsulfanyl (i.e., where R¹² is methyl), ethylsulfanyl, and ethylsulfanyl (i.e., where R¹² is ethyl), propylsulfonyl, and propylsulfanyl (i.e., where R¹² is propyl) and the like.

“Hydroxy protecting group” refers to protecting groups for an OH group. The term, as used herein, also refers to protection of the OH group of an acid COON. Suitable hydroxy protecting groups, as well as suitable conditions for protecting and deprotecting particular functional groups are well-known in the art. For example, numerous such protecting groups are described in T. W. Greene and P. G. M. Wuts, Protecting Groups in Organic Synthesis, Third Edition, Wiley, NY (1999), Such hydroxy protecting groups include C₁₋₆alkyl ethers, benzyl ethers, p-methoxybenzyl ethers, sityl ethers and the like.

“Optionally substituted” or “substituted” refers to the replacement of one or more hydrogen atoms with a monovalent or divalent radical.

When the substituted substituent includes a straight chain group, the substitution can occur either within the chain (e.g., 2-hydroxypropyl, 2-aminobutyl and the like) or at the chain terminus (e.g., 2-hydroxyethyl, 3-cyanopropyl and the like). Substituted substitutents can be straight chain, branched or cyclic arrangements of covalently bonded carbon or heteroatoms.

It is understood that the above definitions are not intended to include impermissible substitution patterns (e.g., methyl substituted with five fluoro groups or a halogen atom substituted with another halogen atom). Such impermissible substitution patterns are well known to the skilled artisan.

It will also be apparent to those skilled in the art that the compounds of the invention. including the compounds of formula (I), (II), (Ill) or (IV) or their stereoisomers and polymorphs, as well as the pharmaceutically acceptable salts, esters, metabolites and prodrugs of any of them, may be subject to tautomerization and may therefore exist in various tautomeric forms wherein a proton of one atom of a molecule shifts to another atom and the chemical bonds between the atoms of the molecules are consequently rearranged. See, e.g., March, Advanced Organic Chemistry: Reactions. Mechanisms and Structures, Fourth Edition, John Wiley & Sons, pp. 69-74 (1992).

As used herein, the term “pharmaceutically acceptable salts” refers to the nontoxic acid or alkaline earth metal salts of the compound, tautomer, stereoiosmer, poiymorph, ester, metabolite or prodrug of formula (I), (II), (Ill) or (IV). These salts can be prepared in situ during the final isolation and purification of the compounds of formulas (I), (II), (III) or (IV), or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quatemized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, phenyl alkyl halides like benzyl and phenethyl bromides, and others, Water or oil-soluble or dispersible products are thereby obtained.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations including, but not limited to ammonium, tetramethylammonium, tetraethylammonium, methylamine, dimethylamine, trimethylamine, triethylamine, ethylamine and the like. Other representative organic amines useful for the formation of base addition salts include diethylamine, ethylenediamine, ethanolamine, diethanolamine, piperazine and the like.

In one embodiment the Raf inhibitor is 1-methyl-5-[2-(5-trifluoromethyl-1H-imidazol-2-yl)-pyridin-4-yloxy]-1H-benzoimidazol-2-yl}-(4-trifluoromethylphenyl)-amine having the following chemical formula:

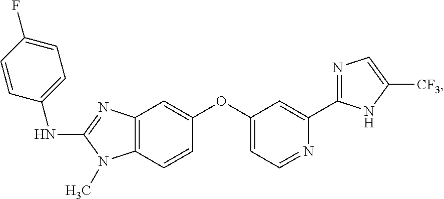

or a pharmaceutically acceptable salt thereof.

In one embodiment, the Raf inhibitor is combined with a platin compound, more specifically cis-platin, for the treatment of PTC. A non-limiting example of a Raf inhibitor is 1-methyl-5-[2-(5-trifluoromethyl-1H-imidazol-2-yl)-pyridin-4-yloxy]-1H-benzoimidazol-2-yl}-(4-trifluoromethylphenyl)-amine having the following chemical formula:

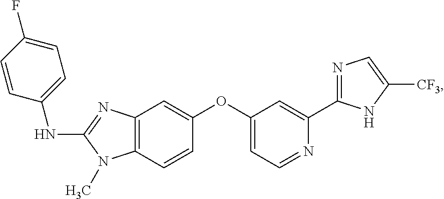

or a pharmaceutically acceptable salt thereof.

“Raf inhibitor” is used herein to refer to a compound that exhibits an IC₅₀ with respect to Rat Kinase activity of no more than about 100 μM and more typically not more than about 50 μM, as measured in the Raf/Mek Filtration Assay described generally hereinbelow, Preferred isoforms of Rat Kinase in which the compounds of the present invention will be shown to inhibit, include A-Raf, B-Raf and C-Raf (Raf-1). “IC₅₀” is that concentration of inhibitor which reduces the activity of an enzyme (e.g., Raf kinase) to half-maximal level. Representative compounds of the present invention have been discovered to exhibit inhibitory activity against Rat. Compounds of the present invention preferably exhibit an IC₅₀ with respect to Raf of no more than about 10 μM, more preferably, no more than about 5 μM, even more preferably not more than about 1 μM, and most preferably, not more than about 200 nM, as measured in the Rat kinase assays described herein.

The compounds of the present invention may be administered orally, parenterally, sublingually, by aerosolization or inhalation spray, rectally or topically in dosage unit formulations containing conventional non-toxic pharmaceutically acceptable carriers, adjuvants and vehicles as desired. Topical administration may also involve the use of transdermal administration, such as transdermal patches or ionophoresis devices. The term “parenteral”, as used herein, includes subcutaneous injections, intravenous, intramuscular, intrasternal injection or infusion techniques.

The person skilled in the pertinent art is fully enabled to select relevant test models to prove the beneficial effects mentioned herein on PTC. The pharmacological activity of such a compound may, e.g., be demonstrated by means of the Examples described below, by in vitro tests and in vivo tests or in suitable clinical studies. Suitable clinical studies are, e.g., open-label, non-randomized, dose escalation studies in patients with PTC. The efficacy of the treatment is determined in these studies. e.g., by evaluation of the tumor sizes every 4 weeks, with the control achieved on placebo.

### EXAMPLE 1

### Effects on MAPK Signaling in Vitro

Studied the effects of Raf inhibitors on MAPK signaling in vitro. Ten cell lines were tested: 5 with BRAF, and 5 with RET/PTC mutations to examine the potential for resistance through inhibition of MAPK phosphatases.

1) Effects on growth, cell cycle and apoptosis.
2) Effects on tumor xenografts: doses 50, 30 and 10 mg/kg/d by gavage.
3) Explore effect of RAF265 in combination with cisplatin in vitro, and in xenografts.

### EXAMPLE 2

The anti-proliferative activity of RAF265 was tested against 4 papillary thyroid carcinoma cell lines, all expressing a luciferase transgene; BHP5-16, BHP14-9, BHP17-10, and NPA87. Cells were seeded into 384 well plates and serial dilutions of RAF265 (e.g., 0.0002-4 μM) was added. The plates were incubated for 2 days at 37° C. Cell proliferation was determined by luciferase expression as measured by Bright-Glo (Promega).

The anti-tumor activity of RAF265 was tested in vivo against the BHP17-10 xenograft model. BHP17-10 cells were implanted subcutaneously into immune-compromised mice and once tumors reach an average volume of approximately 70 mm³, treatment with RAF265 commenced at 100, 30 and 10 mg/kg q3dx5, Tumor volume was measured using calipers 2-3 times weekly. The anti-tumor effect of RAF265 was determined relative to a vehicle-treated control.

1.-12. (canceled)

13. A method of treating papillary thyroid cancer comprising administering a therapeutically effective amount of 1 methyl-5-[2-(5-trifluoromethyl-1H-imidazol-2-yl)-pyridin-4-yloxy]-1H-benzoimidazol-2-yl}-(4-trifluoromethylphenyl)-amine or a tautomer, or a pharmaceutically acceptable salt thereof to a warm-blooded animal in need thereof.

14.-23. (canceled)

24. The method according to claim 13, wherein the wan 1-blooded animal is a human.

25. A method of treating papillary thyroid cancer comprising administering a therapeutically effective amount of a Raf inhibitor to a warm-blooded animal in need thereof in combination with cisplatin wherein the Raf inhibitor is I-methyl-5-[2-(5- trifluoromethyl-1H-imidazol-2-yl)-pyridin-4-yloxy]-1H-benzoimidazol-2-yl}-(4-trifluoromethylphenyl)-amine or a tautomer, or a pharmaceutically acceptable salt thereof.
